(19) United States
(12) Patent Application Publication
(10) Pub. No.: US 2025/0261857 A1
(43) Pub. Date: Aug. 21, 2025

(54) VIRTUAL FIELD OF VIEW FOR PATIENT MONITORING

(71) Applicant: Welch Allyn, Inc., Skaneateles Falls, NY (US)

(72) Inventors: WonKyung McSweeney, Manlius, NY (US); David E. Quinn, Moravia, NY (US); Gene J. Wolfe, Pittsford, NY (US)

(21) Appl. No.: 19/045,048

(22) Filed: Feb. 4, 2025

Related U.S. Application Data
(60) Provisional application No. 63/555,560, filed on Feb. 20, 2024.

(51) Int. Cl. A61B 5/00; G16H 10/60

(52) U.S. Cl. CPC A61B 5/002; A61B 5/0013; A61B 5/7425; A61B 5/7435; A61B 5/744; A61B 5/7475; G16H 10/60

(57) ABSTRACT

A system for remotely monitoring a patient in a patient environment. The system receives an environment view and determines one or more objects for monitoring in the environment view. The system receives video data of the one or more objects. The video data is captured by a camera positioned inside the patient environment. The system generates a virtual field of view using the video data of the one or more objects. The virtual field of view provides the environment view and detailed views of the one or more objects within the environment view.

### CROSS-REFERENCE TO RELATED APPLICATION

This application claims the benefit of and priority to U.S. Provisional Patent Application No. 63/555,560, filed Feb. 20, 2024, the entire disclosure of which is incorporated by reference herein in its entirety.

### BACKGROUND

Video surveillance is increasingly being used for monitoring patients within healthcare environments such as hospitals in view of caregiver shortages. To encapsulate necessary details for effective remote monitoring of a patient, it is desirable to be able to focus on one or more regions of interest. However, it is also desirable to preserve an overall view of the patient's surroundings for context. Obtaining high-resolution video while maintaining environmental context awareness with limited equipment is challenging.

### SUMMARY

In general terms, the present disclosure relates to remote patient monitoring. In one possible configuration, a virtual field of view is generated that provides an environment view and detailed views of one or more regions of interest within the environment view. Various aspects are described in this disclosure, which include, but are not limited to, the following aspects.

One aspect relates to a system for remotely monitoring a patient in a patient environment, the system comprising: at least one processing device; and at least one computer readable data storage device storing software instructions that, when executed by the at least one processing device, cause the at least one processing device to: receive an environment view; determine one or more objects for monitoring in the environment view; receive video data of the one or more objects, the video data captured by a camera positioned inside the patient environment; and generate a virtual field of view using the video data of the one or more objects, the virtual field of view providing the environment view and detailed views of the one or more objects within the environment view.

Another aspect relates to a method of remotely monitoring a patient in a patient environment, the method comprising: receiving an environment view of the patient environment; determining one or more objects for monitoring in the environment view; receiving video data of the one or more objects, the video data captured by a camera positioned inside the patient environment; and generating a virtual field of view using the video data of the one or more objects, the virtual field of view providing the environment view and detailed views of the one or more objects within the environment view.

Another aspect relates to a non-transitory computer readable storage media including computer readable instructions which, when read and executed by a computing device, cause the computing device to: receive an environment view; determine one or more objects for monitoring in the environment view; receive video data of the one or more objects, the video data captured by a camera positioned inside the patient environment; and generate a virtual field of view using the video data of the one or more objects, the virtual field of view providing the environment view and detailed views of the one or more objects within the environment view.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for monitoring a patient in a patient environment by generating a virtual field of view.

FIG. 2 schematically illustrates an example of a camera that can be implemented in the patient environment of FIG. 1.

FIG. 3 schematically illustrates an example of a method of capturing video data that can be used for generating the virtual field of view of the patient environment of FIG. 1.

FIG. 4 schematically illustrates an example of a method of generating the virtual field of view of the patient environment of FIG. 1.

FIG. 5 illustrates an example of the virtual field of view that can be generated by the method of FIG. 4.

FIG. 6 schematically illustrates an example of a method of operating the virtual field of view of FIG. 5.

### DETAILED DESCRIPTION

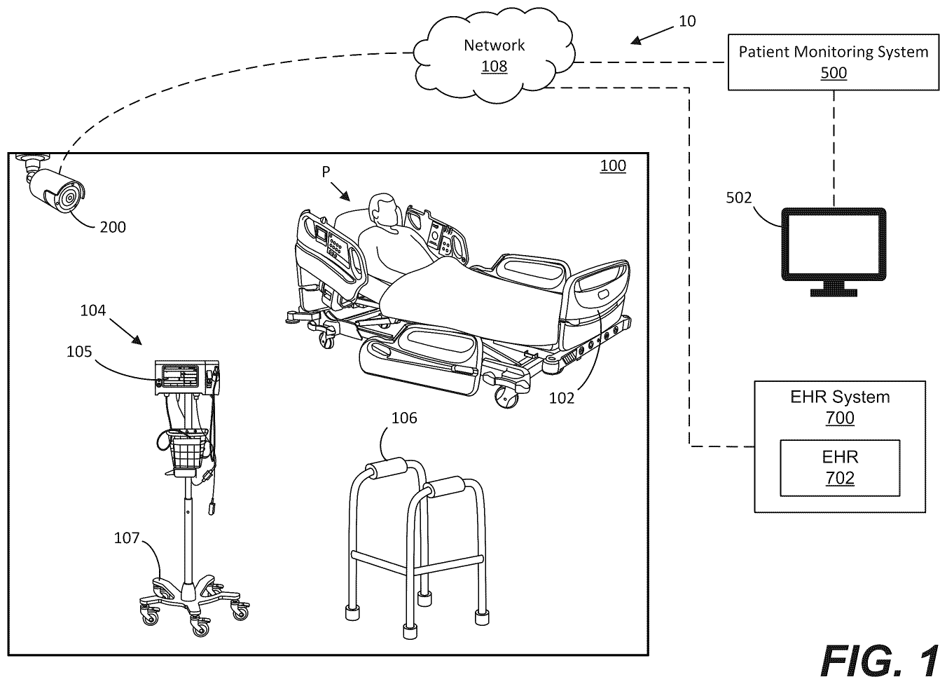

FIG. 1 illustrates an example of a system 10 for monitoring a patient P in a patient environment 100 by generating a virtual field of view. The patient P is shown resting on a patient support apparatus 102 inside the patient environment 100. The patient environment 100 can be an area within a medical facility such as a patient room in a hospital. The patient environment 100 includes medical equipment such as the patient support apparatus 102, and other medical equipment such as a patient monitoring device 104, and an ambulation device 106.

As shown in FIG. 1, the patient P is supported on the patient support apparatus 102 inside the patient environment 100. The patient support apparatus 102 can be a hospital bed, a stretcher, operating room table, or similar type of apparatus on which the patient P can rest. The patient support apparatus 102 can include one or more sensors that measure one or more physiological parameters of the patient P such as heart rate, non-invasive blood pressure (NIBP), motion, and weight. Additionally, the patient support apparatus 102 can include sensors that detect patient exit, incontinence, deterioration, and other metrics.

The patient monitoring device 104 can be used to measure and monitor physiological parameters of the patient P, and to display representations of the measured physiological parameters on a display 105. The display 105 can include a touchscreen that operates to receive tactile inputs from a user such as a caregiver such that the display 105 is both a display device and a user input device. In some examples, the display 105 is a liquid-crystal display (LCD), an organic light-emitting diode (OLED, a plasma panel, a quantum-dot light-emitting diode (QLED), or other type or combination of display screen technology.

The patient monitoring device 104 includes one or more sensor modules that can be used to measure one or more physiological parameters of the patient P. For example, the patient monitoring device 104 can include a temperature sensor module for measuring the patient P's temperature, a pulse oximetry sensor module for measuring the patient P's blood oxygen saturation (SpO2), and a non-invasive blood pressure (NIBP) sensor measurement module for measuring the patient P's blood pressure. As used herein, a “module” is a combination of physical structure which resides in the patient monitoring device 104 and peripheral components that attach to and reside outside of the patient monitoring device 104. The patient monitoring device 104 can include additional sensor modules for receiving additional physiological parameter measurements, including heart rate, pulse, and ECG/EKG.

In the illustrative example shown in FIG. 1, the patient monitoring device 104 is mounted on a mobile cart 107 such that the patient monitoring device 104 is portable and can be brought into and out of the patient environment 100. In alternative examples, the patient monitoring device 104 can be stationary such that it can include a wall mounted unit.

Also, in the example shown in FIG. 1, the ambulation device 106 is illustrated as a walker. Additional types of ambulation devices are contemplated.

As shown in FIG. 1, the system 10 includes a camera 200 that is mounted to a wall of the patient environment 100. The camera 200 can be mounted at different locations within the patient environment 100. In alternative examples, the system 10 can include a plurality of cameras mounted onto the walls or elsewhere within the patient environment 100.

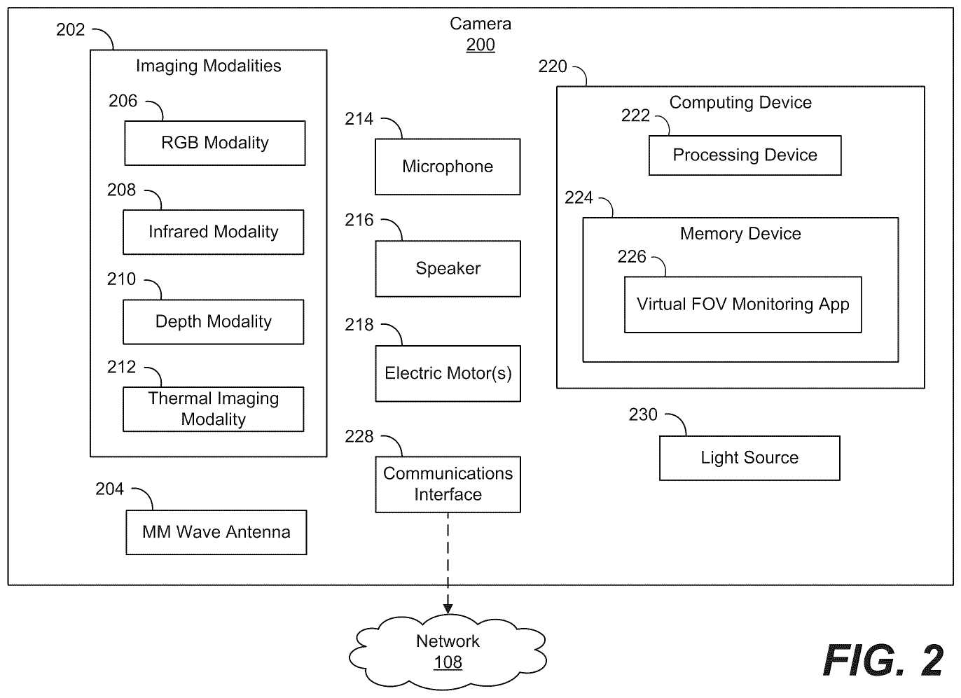

The camera 200 is configured to pan, tilt, and zoom for adjusting a view of the patient environment 100 as well as views of individual objects within the patient environment 100 such as the patient P, the patient support apparatus 102, the patient monitoring device 104, and the ambulation device 106. The camera 200 can include a gimbal or similar structure that is actuated by an electric motor 218 (see FIG. 2) to pan the camera 200 between left and right and to tilt the camera 200 up and down. Also, the camera 200 can zoom in and out by adjusting a focal length of a lens whether by mechanically (e.g., mechanical zoom) or digitally (e.g., digital zoom).

The camera 200 operates to capture a wide field of view of the patient environment 100 that can range across 180 degrees, or more. Also, the camera 200 operates to zoom-in on one or more regions of interest within the patient environment 100 such as a region of interest on the patient P, a region of interest on the patient support apparatus 102, a region of interest on the patient monitoring device 104, or a region of interest on the ambulation device 106. As will be described in more detail, the camera 200 is controlled to switch between the wide field of view and the views of the regions of interest based on a timed interval or a detected trigger event.

Video data captured by the camera 200 can be used to extract one or more parameters relevant to remote monitoring of the patient P. For example, physiological parameters of the patient P such as heart rate, respiration rate, patient movement, and the like can be extracted from the video data captured by the camera 200. Several techniques can be used to determine the respiration rate of the patient P from the video data captured by the camera 200, such as those described in U.S. Provisional Patent Application No. 63/489,901, filed Mar. 13, 2023, entitled Respiration Monitoring, which is incorporated herein by reference in its entirety.

Additional parameters can be extracted from the video data captured by the camera 200 such as a state of operation or functioning of the patient support apparatus 102, the patient monitoring device 104, and/or the ambulation device 106. For example, the camera 200 can zoom-in on the patient support apparatus 102 to determine one or more settings or states of operation of the patient support apparatus 102 such as a bed angle displayed on a display of the patient support apparatus 102. As another example, the camera 200 can zoom-in on the patient monitoring device 104 to determine a state of operation such as whether one or more alarms are triggered on the display 105 of the patient monitoring device 104, or one or more vital sign measurements that are displayed on the display 105 of the patient monitoring device 104.

As shown in FIG. 1, the camera 200 is connected to a network 108. In some examples, the network 108 connects and exchanges data between the camera 200 and other equipment inside the patient environment 100, as well as between the camera and other systems and devices outside of the patient environment 100. The network 108 can include any type of wired or wireless connections, or any combinations thereof. In some examples, the wireless connections can be accomplished using Wi-Fi, ultra-wideband (UWB), Bluetooth, and the like. In some examples, the network 108 is an Internet of things (IoT) network.

As shown in FIG. 1, the network 108 transfers the data captured by the camera 200 to a patient monitoring system 500 for display on a workstation monitor 502. The network 108 provides two-way communications between the patient monitoring system 500 and the camera 200. In some examples, the patient monitoring system 500 is part of a nurse call system. As will be described in more detail, the patient monitoring system 500 displays a virtual field of view on the workstation monitor 502 based on the video data captured by the camera 200.

In some examples, the patient monitoring system 500 is communicatively connected to the workstation monitor 502 via the network 108. Alternatively, the patient monitoring system 500 can be connected directly to the workstation monitor 502 via wired and/or wireless connections without using the network 108 to communicate with the workstation monitor 502.

As further shown in FIG. 1, video data captured by the camera 200 including parameters that are determined from analysis of the video data can be transferred over the network 108 to an EHR system 700 for storage in an electronic health record (EHR) 702. As described herein, the terms electronic medical records (EMRs) and electronic patient record (EPRs) can be used interchangeably with EHRs. The EHR system 700 collects patient electronically stored health information in a digital format (e.g., EHRs 702). As such, the EHR system 700 maintains a plurality of EHRs 702 for a plurality of patients. Each EHR 702 can be shared across different health care settings. For example, the EHRs 702 are shared through network-connected, enterprise-wide information systems or other information networks and exchanges. The EHRs 702 may include a range of data, including demographics, medical history, medication and allergies, immunization status, laboratory test results, radiology images, vital signs, personal statistics like age and weight, and billing information.

FIG. 2 schematically illustrates an example of the camera 200 that can be implemented in the patient environment 100 of FIG. 1. The camera 200 includes one or more types of imaging modalities 202. For example, the imaging modalities 202 include an RGB imaging modality 206 that can be used to capture a color video of the patient environment 100 and of regions of interest on the objects located inside the patient environment 100.

The imaging modalities 202 of the camera 200 can further include an infrared imaging modality 208 that can be used to capture an infrared video of the patient environment 100 and of the objects that are located inside the patient environment 100. The imaging modalities 202 of the camera 200 can also include a depth imaging modality 210 that can be used to capture depth (D) data of the objects that are located inside the patient environment 100. The imaging modalities 202 can also include a thermal imaging modality 212 that can be used to capture a thermal imaging video of the patient environment 100 and of the objects that are located inside the patient environment 100. The foregoing examples of the imaging modalities 202 are not exhaustive, and it is contemplated that the camera 200 can include additional types of imaging modalities 202 for capturing video data of the patient environment 100 and of the objects that are located inside the patient environment 100.

As further shown in FIG. 2, the camera 200 can also include a millimeter wave antenna 204 that can be used to detect movements within the patient environment 100 such as motion by the patient P and/or other persons such as caregivers inside the patient environment 100. The millimeter wave antenna 204 can also detect movements of equipment inside the patient environment 100 such as movements of the patient support apparatus 102, the patient monitoring device 104, and the ambulation device 106. Additionally, the millimeter wave antenna 204 can also detect motion such as chest movements for calculating a non-contact vital sign measurement based on the reflected millimeter wave signals of the patient P. The millimeter wave antenna 204 can include aspects described in U.S. Pat. No. 11,653,848 B2, granted on May 23, 2023, which is incorporated herein by reference in its entirety. In some examples, an imaging modality 202 of the camera 200 and the millimeter wave measurements by the millimeter wave antenna 204 can occur simultaneously on the camera 200.

The camera 200 can further include a microphone 214 that can detect audio inside the patient environment 100. For example, the microphone 214 can detect noises due to movement of the patient P, or movement of one or more pieces of equipment inside the patient environment 100 such as by the patient support apparatus 102, the patient monitoring device 104, or the ambulation device 106. While FIG. 2 shows the microphone 214 integrated with the camera 200, in alternative examples, the microphone 214 can be a separate device that is located outside of the camera 200, and positioned elsewhere in the patient environment 100. In some examples, the microphone 214 can be integrated with other pieces of equipment inside the patient environment 100 that are communicatively coupled to the patient monitoring system 500 such as the patient support apparatus 102, the patient monitoring device 104, or the ambulation device 106.

In some examples, the camera 200 includes a speaker 216 that can be controlled by the patient monitoring system 500 to provide audio for interaction with the patient P inside the patient environment 100. For example, when the patient P is under a blanket such that a view of the patient P's chest is obstructed, the patient monitoring system 500 can emit an audio command through the speaker 216 to communicate to the patient P the need to remove the blanket so that the camera 200 can obtain an unobstructed view of the patient P's chest. As another illustrative example, when the patient P is moving inside the patient environment 100, the patient monitoring system 500 can emit an audio command through the speaker 216 to communicate to the patient P the need to remain still such that a non-contact vital sign measurement can be calculated based on the video data captured by the camera 200 or based on the reflected millimeter wave signals captured by the millimeter wave antenna 204.

While FIG. 2 shows the speaker 216 integrated with the camera 200, in alternative examples, the speaker 216 can be a separate device that is located outside of the camera 200, and positioned elsewhere in the patient environment 100. In some further examples, the speaker 216 can be integrated with other pieces of equipment inside the patient environment that are communicatively coupled to the patient monitoring system 500 such as the patient support apparatus 102, the patient monitoring device 104, or the ambulation device 106.

As further shown in FIG. 2, the camera 200 includes a computing device 220 having at least one processing device 222 and a memory device 224. The at least one processing device 222 is an example of a processing unit such as a central processing unit (CPU). The at least one processing device 222 can include one or more central processing units (CPUs). In some examples, the at least one processing device 222 includes one or more digital signal processors, field-programmable gate arrays, and/or other types of electronic circuits.

The memory device 224 operates to store data and instructions for execution by the at least one processing device 222. In the example illustrated in FIG. 2, the memory device 224 stores a virtual field of view monitoring application 226, which will be described in more detail. The memory device 224 includes computer-readable media, which may include any media that can be accessed by the camera 200. By way of example, computer-readable media include computer readable storage media and computer readable communication media. As such, the memory device 224 is an example of a computer readable data storage device storing software instructions for execution by the at least one processing device 222.

Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the camera. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The camera 200 further includes a communications interface 228 that allows the camera 200 to connect to the network 108. The communications interface 228 can include wired interfaces and/or wireless interfaces. For example, the communications interface 228 can wirelessly connect to the network 108 through Wi-Fi, or other wireless connections. Alternatively, the communications interface 228 can connect to the network 108 using wired connections such as through an Ethernet or Universal Serial Bus (USB) cable.

As further shown in FIG. 2, the camera 200 includes one or more electric motors 218. The one or more electric motors are controlled to pan the camera 200 between left and right and to tilt the camera 200 up and down. Also, the one or more electric motors 218 can be used to adjust a focal length of a lens of the camera 200 to provide mechanical zoom adjustment.

The camera 200 further includes a light source 230 that is controlled to illuminate a region of interest in the patient environment 100. For example, the light source 230 can generate a spotlight to illuminate with white light a region of interest for capturing a color video of the region. In further examples, the light source 230 can emit a referential dot, shape, or pattern that can be tracked in the videos captured by the camera 200 to measure one or more parameters such as respiration rate that is determined based on movement of the patient P's chest.

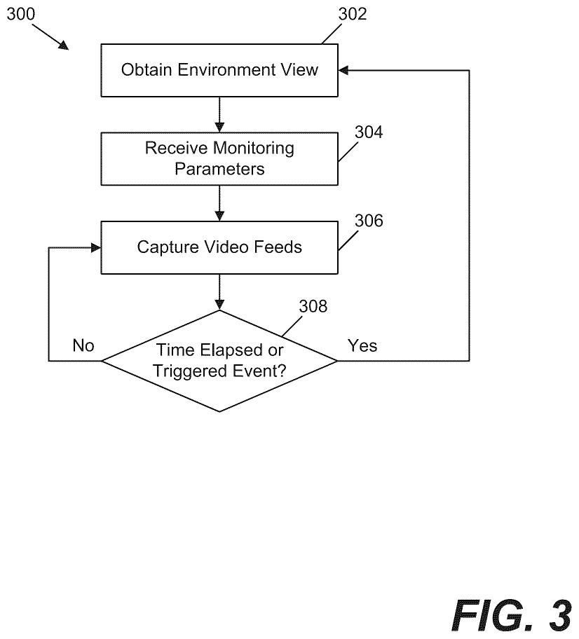

FIG. 3 schematically illustrates an example of a method 300 of capturing video data that can be used for generating a virtual field of view of the patient environment 100. The method 300 can be performed by the camera 200. The method 300 includes an operation 302 of obtaining an environment view of the patient environment 100. The environment view is a wide field of view of the patient environment 100. For example, the environment view can span across a range of 180 degrees or more such that the patient P and all other objects and pieces of equipment inside the patient environment 100 are included in the environment view.

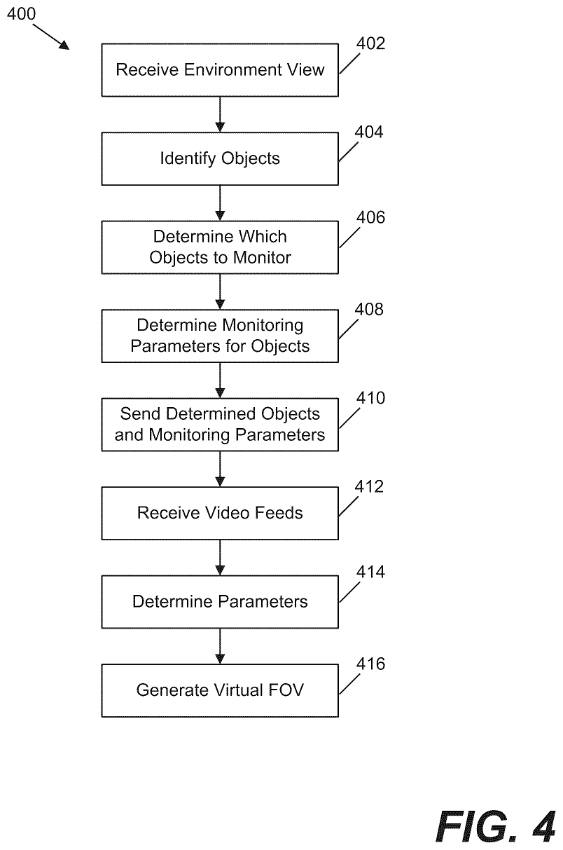

The method 300 includes an operation 304 of receiving monitoring parameters that are determined based on the environment view captured in operation 302. In some examples, the monitoring parameters are determined by the patient monitoring system 500, and are received by the camera 200 from the patient monitoring system 500 over the network 108. In alternative examples, the camera 200 can determine the monitoring parameters based on the environment view captured in operation 302. The monitoring parameters will be described in more detail further below with reference to the method 400 of FIG. 4.

The method 300 includes an operation 306 of capturing video feeds of one or more regions of interest based on the monitoring parameters received in operation 304. The video feeds can include color videos using the RGB imaging modality 206, infrared videos using the infrared imaging modality 208, depth videos using the depth imaging modality 210, and/or thermal videos using the thermal imaging modality 212.

Operation 306 can include capturing the video feeds based on a predetermine sequence such as a first video feed of a first region of interest, followed by a second video feed of a second region of interest, and so on. Also, the video feed of each region of interest can be captured by the camera 200 using a different imaging modality 202 based on the monitoring parameters received in operation 304. This can mitigate computational burdens on the camera 200 and the patient monitoring system 500 because certain imaging modalities may have higher data processing and data transfer bandwidth burdens than other imaging modalities. For example, the depth imaging modality 310 typically has a higher data processing and transfer bandwidth burden than other imaging modalities. By capturing the video feeds in a predetermined sequence such that the depth imaging modality 310 is used periodically by the camera 200 (instead of continuously), the data processing and data transfer bandwidth burdens on the camera 200 are mitigated, thereby improving the functional performance of the camera 200.

As an illustrative example, operation 306 can include capturing a video feed of the patient P's chest that can be used to measure the patient P's respiration rate. As another example, operation 306 can further include capturing a video feed of a display on the patient support apparatus 102 that can be used to view one or more settings or states of operation of the patient support apparatus 102 such as a bed angle. As another example, operation 306 can include capturing a video feed of the display 105 on the patient monitoring device 104 to view the physiological variable measurements captured by the patient monitoring device 104, or whether one or more alarms are triggered on the patient monitoring device 104.

In some examples, when a region of interest cannot be viewed by the camera 200 such as due to an obstruction, operation 306 can include issuing an audible command through the speaker 216 to instruct the patient P or another person such as a caregiver inside the patient environment 100 to remove the obstruction. For example, when a region of interest includes the patient P's chest which is obscured by a blanket, operation 306 can include issuing an audible command through the speaker 216 to instruct the patient P or another person to remove the blanket from the patient P's chest to allow the camera 200 to capture a video feed of the chest.

The method 300 includes an operation 308 of determining whether a predetermined time period has elapsed or a trigger event has occurred. When operation 308 determines that the predetermined time period has not elapsed and one or more trigger events have not occurred (i.e., “No” in operation 308), the method 300 continues the operation 306 of capturing the one or more video feeds. When operation 308 determines that the predetermined time period has elapsed or one or more trigger events have occurred (i.e., “Yes” in operation 308), the method 300 returns to operation 302 to obtain an environment view of the patient environment 100.

As an illustrative example, a trigger event can include an event or measurement detected based on a video feed captured in operation 306. For example, a video feed of a region of interest on the patient P can be analyzed in operation 308 to detect an event, which causes the method 300 to return to operation 302 such that the camera 200 zooms out to capture the environment view of the patient environment 100. This can provide context to explain a potential cause of the event detected in the video feed of the region of interest on the patient P.

As an illustrative example, operation 308 can include analyzing a video feed of a region of interest on the patient P's chest that detects a decrease in respiration rate by the patient P. This causes the method 300 to return to operation 302 such that the camera 200 zooms out to capture the environment view of the patient environment 100, which shows the patient P is talking with another person who has entered the patient environment 100. This provides context to explain the decrease in respiration rate by the patient P such that an alarm that would otherwise be triggered by the patient monitoring system 500 is suppressed.

As another illustrative example, a trigger event can include a sound detected by the microphone 214 that indicates movement of the patient P within the patient environment 100 such as when the patient P exists the patient support apparatus 102. As another example, a trigger event can include a sound detected by the microphone 214 that indicates an event such as a patient fall. A trigger event can also include a sound detected by the microphone 214 that indicates movement of an object within the patient environment 100. As another example, a trigger event can include a sound detected by the microphone 214 that indicates an audible alarm triggered on one or more pieces of equipment inside the patient environment 100 such as an alarm triggered on the patient support apparatus 102 or the patient monitoring device 104.

In this manner, the method 300 updates the environment view of the patient environment 100 to maintain overall surveillance and awareness of the patient environment 100, while also obtaining video feeds of particular regions of interest within the patient environment 100. This can be accomplished by using a single camera, instead of requiring use of a plurality of cameras, and can thereby improve efficiencies within the medical facility where the patient environment 100 is located by reducing the need to employ additional cameras. The method 300 can improve the functioning of the camera 200 by allowing the camera 200 to capture detailed images or videos of one or more regions of interest on objects within the patient environment 100 without sacrificing a broader contextual view of the patient environment 100.

FIG. 4 schematically illustrates an example of a method 400 of generating a virtual field of view of the patient environment 100. The method 400 can be performed by the patient monitoring system 500. In some examples, aspects of the method 400 can be performed on the camera 200. The method 400 includes an operation 402 of receiving the environment view from the camera 200. The environment view can be received over the network 108.

The method 400 includes an operation 404 of identifying objects in the environment view. The objects identified in operation 404 depend on the persons and equipment located inside the patient environment 100 at the time the environment view is captured by the camera 200. For example, in view of the patient environment 100 shown in FIG. 1, operation 404 can include identifying the patient P, the patient support apparatus 102, the patient monitoring device 104, and the ambulation device 106. Operation 404 includes identifying the objects using one or more algorithms such as artificial intelligence and machine learning algorithms.

The method 400 includes an operation 406 of determining which objects to monitor from the objects identified in operation 404. In some examples, operation 406 can include determining which objects to monitor based on whether an object is stationary, or is non-stationary such that it is portable or movable within the patient environment 100. Stationary objects like walls and doors remain unchanged and do not require periodic updates. Conversely, portable or movable objects including the patient P and items that can be manipulated by the patient P or caregivers (e.g., the patient support apparatus 102, the patient monitoring device 104, the ambulation device 106, intravenous (IV) pumps, and the like) are dynamic such that they should be periodically monitored by adjusting the viewpoint of the camera 200.

In some examples, operation 406 can include applying one or more flags to the objects identified in operation 404. For example, operation 406 can include applying a dynamic flag on the patient monitoring device 104 when mounted on the mobile cart 107 because the patient monitoring device 104 is portable. In examples where the patient monitoring device 104 is wall mounted such that it is not portable, operation 406 can include applying a static flag to the patient monitoring device 104. The dynamic/static flags can be used to determine whether to select an object for periodic monitoring or not. For example, static objects are not selected for periodic monitoring since they are stationary and their position does not change within the patient environment 100. In contrast, portable objects can be selected for periodic monitoring since the position of these objects can change within the patient environment 100.

In some examples, operation 406 can include determining which objects to monitor is based on a condition identified in the environment view received in operation 402. As an illustrative example, operation 406 can include selecting the ambulation device 106 as an object to monitor when the environment view shows the patient P as being within a predetermined distance of the ambulation device 106 such that patient P is within reach of using the device.

In some examples, operation 406 includes determining which objects to monitor based on information acquired from the EHR system 700. For example, operation 406 can include determining which objects to monitor based on one or more diseases, conditions, and/or diagnoses of the patient P stored in the EHR 702. As an illustrative example, operation 406 can include selecting the patient P as an object to monitor when the EHR 702 identifies the patient P as having a fall risk, or as having a health condition such that it is recommended to measure physiological variables such as respiration rate through non-contact means. As another illustrative example, operation 406 can include selecting an infusion pump as an object to monitor when the EHR 702 identifies the patient P is undergoing an infusion therapy.

The method 400 includes an operation 408 of determining one or more monitoring parameters for the objects determined in operation 406. The monitoring parameters can define, without limitation, a region of interest on the object, a minimum resolution, a minimum field of view, a movement tolerance, a minimum illumination, and a monitoring frequency. The monitoring parameters control the movement and positioning of the camera 200 when targeting an object during the periodic monitoring of the object performed in the method 300 of FIG. 3.

As an illustrative example, when operation 406 selects the patient P as an object for monitoring the respiration rate of the patient P, operation 408 can include defining the chest as the region of interest, a minimum resolution of 200×200 pixels/inch, a minimum field of view that includes the patient P's full body with a minimum allowed obstruction of 20%, a movement tolerance of 0 meters/second, a lighting requirement of 0.01 Lux, and a monitoring frequency of 3 minutes when respiration rate measurements are within a normal range.

As another example, when operation 406 selects the patient monitoring device 104 as an object for monitoring in the patient environment 100, operation 408 can include defining the display 105 as the region of interest, and can further select a minimum resolution and a minimum frequency for capturing a video of the display 105 on the patient monitoring device 104.

The method 400 includes an operation 410 of sending to the camera 200 the objects determined in operation 406 and the monitoring parameters determined in operation 408. Operation 410 can be performing using the network 108 which communicatively connects the patient monitoring system 500 to the camera 200.

The method 400 includes an operation 412 of receiving the video feeds from the camera 200. The video feeds are captured by the camera 200 based on the objects determined in operation 406 and the monitoring parameters determined for objects in operation 408. The video feeds can be received from the camera 200 over the network 108.

The method 400 can include an operation 414 of determining one or more physiological parameters based on the video feeds received in operation 412. For example, operation 414 can include determining the respiration rate of the patient P based on the up and down chest movements observed in a video feed of the patient P's chest.

The method 400 includes an operation 416 of generating a virtual field of view using the video feeds received from the camera 200 in operation 412. The virtual field of view allows a caregiver who is remotely located with respect to the patient environment 100 to simultaneously view a wide field of view of the patient environment 100 (e.g., the environment view) and also detailed views of one or more regions of interest on objects in the patient environment such as a detailed view of the patient P or a detail view of the display 105 on the patient monitoring device 104 without interrupting the monitoring of the patient environment 100 by a single camera.

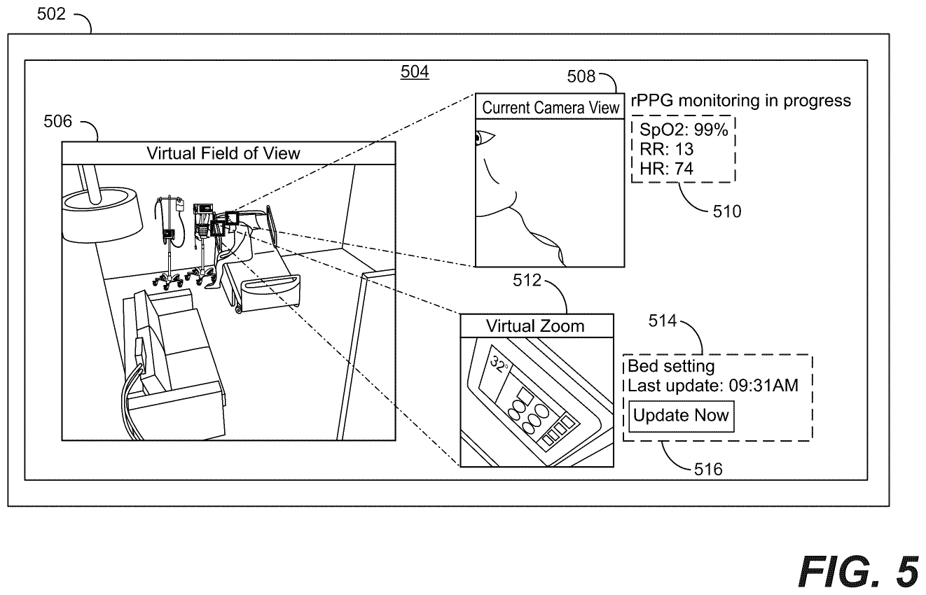

FIG. 5 illustrates an example of a virtual field of view 504 that can be generated in operation 416 of the method 400. In FIG. 5, the virtual field of view 504 is shown as being displayed on the workstation monitor 502. The virtual field of view 504 includes an environment view 506 that is a wide field of view of the patient environment 100. The environment view 506 is captured by the camera 200 in operation 302 of the method 300.

In some examples, the environment view 506 is a simulation based on the video data captured by the camera 200. For example, the actual video of the patient P can be replaced by an avatar that represents the positioning and movements of the patient P. The avatar conceals the patient P's identity, which can mitigate unauthorized access to protected health information.

One or more areas are selectable within the environment view 506 to view a detailed view of a region of interest in the patient environment 100. In the example shown in FIG. 5, a region of interest on the patient P such as the patient P's face is selectable in the environment view 506 to view a detailed view 508 of the patient P within the virtual field of view 504.

The detailed view 508 is a high-resolution image or video that captures details of the patient P, such as facial characteristics and skin tone, which are not visible in the environment view 506. The detailed view 508 can include physiological parameters 510 that are determined from the video feeds captured by the camera 200 in accordance with operation 414 of the method 400. In the illustrative example shown in FIG. 5, the physiological parameters 510 include blood oxygen saturation (SpO2), respiration rate, and heart rate.

In the example shown in FIG. 5, the detailed view 508 indicates that remote photoplethysmography (rPPG) monitoring is in progress. rPPG measures the variance of red, green, and blue light reflection changes from the skin of a region of interest on the patient P where a high density of blood vessels are concentrated such as on the patient P's face. The heart rate and heart rate variability of the patient P are estimated by measuring inter-beat intervals between peaks of the light reflection changes over time.

As another illustrative example, the blood oxygen saturation of the patient P can be calculated based on blood perfusion detected from changes in skin tone in the video feed of the patient P's face. As another illustrative example, the respiration rate and/or heart rate of the patient P can be calculated from measuring chest movements either in a video feed of the patient P's chest, or based on reflected millimeter wave signals captured by the millimeter wave antenna 204. In alternative examples, the physiological parameters 510 can be measured by one or more sensors on the medical equipment located inside the patient environment 100 such as by one or more sensors on the patient support apparatus 102 and/or the patient monitoring device 104.

As another illustrative example, a region of interest on the patient support apparatus 102 is selectable in the environment view 506 to view a detailed view 512 of a display of the patient support apparatus 102. The detailed view 512 is a high-resolution image or video that captures details displayed on the display of the patient support apparatus 102 such as one or more settings of the patient support apparatus 102 such as bed angle, and the like. Such details are not visible in the environment view 506. The detailed view 512 can further include an area 514 that identifies one or more settings of the patient support apparatus 102.

In some examples, the detailed views 508, 512 are simulations based on the video data captured by the camera 200. For example, the actual video of the regions of interest can be replaced by simulations of the regions of interest that are representative of the regions of interest based on the video data captured by the camera 200. The simulations can further conceal the protected health information that is captured by the camera 200.

When a region of interest is selected in the environment view 506 to display a detailed view 508, 512, the operation of the camera 200 is not interrupted because the high-resolution image or video displayed in the detailed view 508, 512 is not necessarily a live image or video feed. Instead, the detailed view 508, 512 can include a display of a previously captured image or video that is captured in accordance with the operations of the method 300.

The image or video displayed in the environment view 506 is current even when it is not a live feed based on the operations of the method 300. For example, when operation 308 includes determining that a predetermined time period has elapsed or a trigger event has occurred, the method 300 returns to operation 302 of recapturing the environment view 506.

Similarly, the images or videos displayed in the detailed views 508, 512 are current even when they are not based on live feed because the method 300 repeats the operation 304 of receiving updated monitoring parameters based on recapturing the environment view 506, and thereafter, the method 300 repeats the operation 306 of recapturing one or more video feeds for display in the detailed views 508, 512 based on the updated monitoring parameters.

The virtual field of view 504 does not continuously monitor all of the regions of interest simultaneously within the patient environment 100. Instead, the detailed views 508, 512 of the regions of interest are updated periodically at predetermined intervals or when a trigger event is detected. Thus, the detailed views 508, 512 do not display real-time video feeds.

Instead, the virtual field of view 504 includes the detailed views 508, 512 as a patchwork of video feeds and/or images captured by the camera 200. In some examples, the video feeds and/or images are captured at different time points. In this manner, the virtual field of view 504 provides dynamic resolution on one or more objects or areas relevant to the healthcare of the patient P inside the patient environment 100. The one or more objects or areas can be highlighted inside the environment view 506 to indicate their relevance to a user of the patient monitoring system 500. The user can select the one or more objects or areas in the environment view 506 to view in the detailed views 508, 512 detailed information such as detailed videos, images, and other data. In this manner, the virtual field of view 504 provides a 3-dimentional space in the environment view 506 where a user of the patient monitoring system 500 can navigate to view specific information, and when desirable, can zoom in on an object or area to display detailed information (as a live or stored feed) in a detailed view 508, 512 such as 3D images or videos, 2D images or videos, infrared images or videos, thermal images or videos, audio information, or textual information relevant to the healthcare provided to the patient P.

In some examples, the detailed views in the patchwork of video feeds include a time stamp to indicate when the image, video, or other data displayed in the detailed view was last updated. A user of the virtual field of view 504 can select the regions of interest in the environment view 506 to zoom in and out, while the camera 200 continues to capture the video feeds (see operation 306 of the method 300) according to a predetermined scheduled which is based on the objects selected for monitoring (see operation 406 of the method 400) and the monitoring parameters selected for each object (see operation 408 of the method 400).

The detailed views in the patchwork of video feeds in the virtual field of view 504 can further include search and playback. For example, each of the detailed views 508, 512 within the environment view 506 can be rewound to a specific time stamp to view a trigger event. The virtual field of view 504 can also zoom out to playback a whole view of the patient room in the environment view 506 at the time stamp of the trigger event in the detailed view to show what else was happening in the room during the trigger event. In such examples, the virtual field of view 504 includes a multi-dimensional video. Metadata is created based on detected trigger events and associated time stamps. The metadata can be searched for playback of one or more detailed views and/or the environment view 506 within the virtual field of view 504. The metadata allows a user to view the environment view 506 and the one or more detailed views at a common point in time. The metadata can be retrieved to put together a whole picture of a trigger event that includes playback of the environment view 506 and playback of one or more detailed views during occurrence of the trigger event detected within the patient environment 100.

The virtual field of view 504 can further include an input 516 (e.g., “Update Now”). The input 516 is selectable to update the image or video captured by the camera 200 for display inside the detailed view 512. For example, when the input 516 is selected, the patient monitoring system 500 controls the camera 200 such as by panning the camera from left to right or from right to left, and/or tilting the camera 200 up or tilting the camera 200 down, and/or adjusting a zoom of the camera 200 to obtain an updated image or video for display in the detailed view 512.

The virtual field of view 504 improves the functioning of the workstation monitor 502 by providing focus on specific regions of interest within the patient environment 100 while at the same time maintaining an overall view of the patient environment 100 to provide context and overall awareness. As noted above, this is accomplished by using a single camera instead of a plurality of cameras in the patient environment 100, and thereby improves efficiencies.

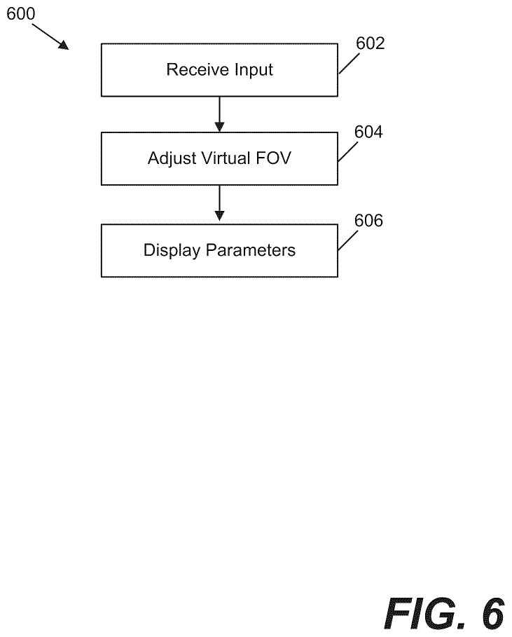

FIG. 6 schematically illustrates an example of a method 600 of operating the virtual field of view 504 shown in FIG. 5. The method 600 can be performed by the patient monitoring system 500 and/or can be performed on the workstation monitor 502. The method 600 includes an operation 602 of receiving an input on the virtual field of view 504. Operation 602 can include receiving a selection of a region of interest in the environment view 506.

The method 600 includes an operation 604 of adjusting the virtual field of view 504 by displaying the region of interest inside a detailed view. Operation 604 is performed without altering the operation of the camera 200 because the region of interest displayed in the detailed view is based on an image or video that is previously captured by the camera 200 and that is considered current or up-to-date based on the operations of the method 300.

The method 600 can include an operation 606 of displaying one or more parameters that are calculated based on the image or video previously captured by the camera 200. For example, operation 606 can include displaying respiration rate, heart rate, and/or SpO2 based on the image or video previously captured by the camera 200. Operation 606 can include displaying the one or more parameters next to the detailed view of the region of interest.

Operation 606 can further include displaying controls to rewind the detailed view of the region of interest to view a trigger event. Further, operation 606 can include zooming out to playback a video stream of the environment view during the occurrence of the trigger event.

Additionally, the benefits of the methods and systems described above can extend to settings that include multiple cameras. As an illustrative example, the patient environment 100 can include a plurality of cameras in which a first camera is configured to focus on a particular object to capture a continuous video feed of the object, while another camera is configured to capture updates of other areas in the patient environment 100. In further examples, the plurality of cameras can include cameras with varying features and operational characteristics such as a wide-angle camera and one or more cameras having a high optical zoom that are independently controllable to pan left and right, tilt up and down, and zoom in and out.

The various embodiments described above are provided by way of illustration only and should not be construed to be limiting in any way. Various modifications can be made to the embodiments described above without departing from the true spirit and scope of the disclosure.

What is claimed is:

1. A system for remotely monitoring a patient in a patient environment, the system comprising:
at least one processing device; and
at least one computer readable data storage device storing software instructions that, when executed by the at least one processing device, cause the at least one processing device to:
receive an environment view;
determine one or more objects for monitoring in the environment view;
receive video data of the one or more objects, the video data captured by a camera positioned inside the patient environment; and
generate a virtual field of view using the video data of the one or more objects, the virtual field of view providing the environment view and detailed views of the one or more objects within the environment view.

2. The system of claim 1, wherein determine the one or more objects for monitoring in the environment view is based on whether the objects are movable within the patient environment.

3. The system of claim 1, wherein determine the one or more objects for monitoring in the environment view is based on a condition identified in the environment view.

4. The system of claim 1, wherein determine the one or more objects for monitoring in the environment view is based on data acquired from an electronic health record system.

5. The system of claim 1, wherein the instructions, when executed by the at least one processing device, further cause the at least one processing device to:
determine monitoring parameters for the one or more objects, the monitoring parameters including one or more of a region of interest, a minimum resolution, a minimum field of view, a movement tolerance, a minimum illumination, and a monitoring frequency.

6. The system of claim 1, wherein the instructions, when executed by the at least one processing device, further cause the at least one processing device to:
determine a physiological parameter measurement based on the video data; and
display the physiological parameter measurement in the virtual field of view.

7. The system of claim 1, wherein the instructions, when executed by the at least one processing device, further cause the at least one processing device to:
display controls to rewind one or more of the detailed views of the one or more objects within the environment view for viewing a trigger event.

8. The system of claim 1, wherein the virtual field of view includes an avatar representing positioning and movements of the patient in the patient environment.

9. A method of remotely monitoring a patient in a patient environment, the method comprising:
receiving an environment view of the patient environment;
determining one or more objects for monitoring in the environment view;
receiving video data of the one or more objects, the video data captured by a camera positioned inside the patient environment; and
generating a virtual field of view using the video data of the one or more objects, the virtual field of view providing the environment view and detailed views of the one or more objects within the environment view.

10. The method of claim 9, wherein determining the one or more objects for monitoring in the environment view is based on whether the objects are movable within the patient environment.

11. The method of claim 9, wherein determining the one or more objects for monitoring in the environment view is based on a condition identified in the environment view.

12. The method of claim 9, wherein determining the one or more objects for monitoring in the environment view is based on data from an electronic health record system.

13. The method of claim 9, further comprising:
determining monitoring parameters for the one or more objects, the monitoring parameters including one or more of a region of interest, a minimum resolution, a minimum field of view, a movement tolerance, a minimum illumination, and a monitoring frequency.

14. The method of claim 9, further comprising:
determining a physiological parameter measurement based on the video data; and
displaying the physiological parameter measurement in the virtual field of view.

15. The method of claim 9, further comprising:
displaying controls to rewind one or more of the detailed views of the one or more objects within the environment view for viewing a trigger event.

16. The method of claim 9, wherein the virtual field of view includes an avatar representing positioning and movements of the patient in the patient environment.

17. A non-transitory computer readable storage media including computer readable instructions which, when read and executed by a computing device, cause the computing device to:
receive an environment view;
determine one or more objects for monitoring in the environment view;
receive video data of the one or more objects, the video data captured by a camera positioned inside the patient environment; and
generate a virtual field of view using the video data of the one or more objects, the virtual field of view providing the environment view and detailed views of the one or more objects within the environment view.

18. The non-transitory computer readable storage media of claim 17, wherein determine the one or more objects for monitoring in the environment view is based on whether the objects are identified as being movable within the patient environment.

19. The non-transitory computer readable storage media of claim 17, wherein determine the one or more objects for monitoring in the environment view is based on a condition identified in the environment view.

20. The non-transitory computer readable storage media of claim 17, wherein determine the one or more objects for monitoring in the environment view is based on data acquired from an electronic health record system.
